(19) United States
(12) Patent Application Publication
(10) Pub. No.: US 2005/0239189 A1
(43) Pub. Date: Oct. 27, 2005

(54) Oxidant-scavenging compound for anaerobic treatment

(76) Inventor: I. Richard Schaffner, Goffstown, NH (US)

Correspondence Address: I. Richard Schaffner, 17 Birch Court, Goffstown, NH, 03045, US

(21) Appl. No.: 10/828,642

(22) Filed: Apr. 21, 2004

(51) Int. Cl. C12S 1/00; C12N 1/18

(52) U.S. Cl. 435/262.5

(57) ABSTRACT

The present invention provides a mixture for use as a biological stimulant in in-situ bioremediation via enhanced reductive dehalogenation. The mixture is primarily a mixture of Lactose and Brewer's Yeast for use as an electron donor in the bioremediation process.

### FIELD OF THE INVENTION

The present invention relates to the field of in-situ bioremediation and, in particular, to enhanced reductive dehalogenation of chlorinated hydrocarbons.

BACKGROUND OF THE INVENTION

Chlorinated solvents are commonly used for commercial applications. Such solvents can include tetrachloroethene (PCE), trichloroethene (TCE), or other chlorinated volatile organic compounds (CVOCs). Common biochemical transformation products of reactant CVOC's include cis/trans 1,2-dichloroethenes (DCEs'), with the cis isomer the most common product, 1,1-DCE, and vinyl chloride (VC). Both parent and daughter CVOC's have been detected in overburden soil and fractured bedrock groundwater systems in urban as well as rural settings for a variety of reasons, ranging from incidental releases to deliberate dumping. CVOC's often persist in groundwater systems owing, at least in part, to their general physical, chemical, and biological properties, such as low aqueous solubility, high specific gravity, and general recalcitrance to natural biological attenuation.

In addition to their persistence in groundwater flow systems, CVOCs pose toxicological risks to both human and animal health. Communities are generally increasing their reliance upon groundwater resources to meet potable water demands. In fact, about one half the population of the United States of America presently uses groundwater to supply potable water. While groundwater is often treated for certain biological and chemical contaminants before distribution, existing treatment technologies at potable water supply facilities are generally incapable of removing significant CVOC mass. Given their toxicity and persistence in groundwater flow systems as well as the limited ability of many water treatment systems to remove them, CVOC's can pose significant risk to groundwater resources for prolonged periods of time.

Given this risk potential, the United States Environmental Protection Agency established Media Cleanup Standards to protect potential sensitive receptors. For CVOC release sites at which potentially responsible parties are required to implement a remedial program to reduce risk to potential sensitive receptors, several remedial technologies are available that can bring sites to closure. Typically, such remedial technologies are selected and implemented as a function of: 1) site hydrogeology; 2) contaminant signature (compound and concentration); 3) costs for engineering design,, capital equipment, construction, operation and maintenance, 4) performance monitoring requirements; 5) present and future property usage; and 6) potential risk to receptors. Groundwater remediation technologies potentially applicable for CVOC releases include, but are not necessarily limited to, the technologies listed below.

In Situ Air Sparging (IAS). Pressurized air is forced into sparge points installed in the saturated zone to enhance CVOC volatilization from both groundwater and the formation matrix. Typically, IAS is combined with soil vapor extraction in the unsaturated zone to recover CVOCs stripped from the saturated zone. Recovered CVOC mass may be discharged into the atmosphere, physically removed by adsorption onto GAC, or biologically/chemically treated depending on local regulatory requirements and stakeholder preferences;

Pump & Treat. Groundwater is extracted and treated via either Air Stripping or Carbon Adsorption. Air Stripping includes passing large fluxes of air through groundwater to volatilize CVOCs. As with IAS, recovered CVOC mass may be discharged to the atmosphere or treated depending on local regulatory requirements and PRP preferences. Carbon Adsorption includes passing groundwater through canisters containing Granular Activated Carbon (GAC). CVOCs adsorb onto the GAC, which is periodically re-activated (i.e., heated at high temperature) to remove CVOCs or replaced with fresh GAC. Spent GAC is disposed either as hazardous or solid waste, depending on CVOC content and PRP preferences;

Chemical Oxidation. Strong chemical oxidizers such as Fenton's Reagent are injected into the contaminant plume to chemically oxidize CVOCs to carbon dioxide, water, and inorganic chloride;

Monitored Natural Attenuation (MNA). The natural processes of adsorption, biodegradation, hydrodynamic dispersion, volatilization, and hydrolysis are monitored to demonstrate they naturally attenuate CVOCs within a reasonable period of time, typically assumed to be about a decade; and

Permeable Reactive Walls. Groundwater flows through a granulated (0 valent) metal treatment wall typically oriented normal to the contaminant plume flow path. CVOCs are destroyed via chemical reactions occurring between CVOCs and granulated metals within the wall.

While many of these remedial technologies have demonstrated effectiveness for managing plume migration (e.g., Pump & Treat, MNA, and permeable reactive walls), several merely transfer CVOCs from one environmental media to another (e.g., Pump & Treat [air stripping] from groundwater to the atmosphere). Furthermore, many technologies are expensive to design, implement, operate, and maintain. Moreover, they have limited effectiveness for treating CVOC source areas, with the exception of IAS and Chemical Oxidation. While IAS and Chemical Oxidation may be effective for source reduction, they are expensive to design, implement, operate, and maintain.

U.S. Pat. No. 5,554,290 to Suthersan discloses the use of carbohydrates for in situ treatment of contaminated groundwater, however, contaminants listed under that patent include metals and nitrate, but not CAHs. Suthersan does not specifically disclose lactose in the formulation.

U.S. Pat. No. 6,150,157 to Keasling, et al. discloses Lactose and Yeast Extract as potential components of a remedial additive for enhancing CVOC reductive dehalogenation. However, the present invention explicitly includes Brewer's Yeast due to the presence of intact cell walls that will reduce the degradation rate of that material, whereas Keasling, et al. specifically discloses Yeast Extract. Additionally, the present invention is formulated to first scavenge terminal electron acceptors from CVOC-impacted groundwater systems, and then drive metabolic reductive dehalogenation once competing oxidants are depleted. Because dehalogenation can only occur in anaerobic groundwater systems devoid of these oxidants, the present invention's formulation prepares the groundwater system for reductive dehalogenation by scavenging competing oxidants. The formulation disclosed by Keasling, et al. is specifically for treating groundwater systems that are generally anaerobic (i.e., mostly devoid of competing oxidants). Notably, Keasling, et al. state “preferred carbohydrates are metabolizable by a broad range of anaerobes.” Additionally, Keasling, et al. claim incubating groundwater under “substantially reducing conditions” (i.e., keeping groundwater anaerobic, not changing redox conditions in a groundwater system).

U.S. Patent Application No. 20020090697 filed to Hince discloses Lactose as a potential component of a remedial additive for enhancing CVOC reductive dehalogenation. The present invention explicitly includes inactive Brewer's Yeast due to the presence of intact cell walls that will reduce the degradation rate of that material, whereas Hince specifically includes an active Yeast culture inoculum. Hince, therefore, includes an active Yeast culture as a bioaugmentation (addition of microbial cultures), not an electron donor and nutrient source as specified in the present invention. Additionally, the present invention is formulated to first scavenge terminal electron acceptors from CVOC-impacted groundwater systems including oxygen, nitrate, ferric iron, and sulfate, and then drive metabolic reductive dehalogenation once competing oxidants are depleted. Because dehalogenation can only occur in anaerobic groundwater systems devoid of these oxidants, the present invention prepares the groundwater system for reductive dehalogenation by scavenging competing oxidants. The formulation disclosed by Hince is specifically for treating groundwater systems that are generally anaerobic (i.e., mostly devoid of competing oxidants) or for scavenging oxygen. For example, Hince indicates that their material was formulated for “creating, enhancing, and maintaining anaerobic if not anoxic conditions by facilitating the biologically mediated removal of the available oxygen from the media. Hince makes no claim that the mixture stimulates nitrate reduction, iron reduction, and sulfate reduction. In fact, Hince specifically includes nitrate as a preferred embodiment of the invention.

Japan Patent Application No. 2003-055606 to Chandraghatgi, Schaffner (present inventor), et al. discloses Lactose and yeast, as well as other additives, for use in the remediation of contaminated soil, groundwater or bottom deposits; however, Chandraghatgi et al. did not specify the form of the yeast in contrast to the present invention that specified Brewer's Yeast. In addition, the present invention specifies the preferred embodiment of 70%_{Weight} Lactose and 30%_{Weight} Brewer's Yeast. Also, the present invention specifies that Lactose is added both to scavenge terminal electron acceptors and to serve as a fermentable source of hydrogen for driving reductive dehalogenation, whereas Chandraghatgi et al. indicate the role of the Lactose is to stimulate aerobic mineralization processes.

What is needed is a remedial technology including Lactose and Yeast that effectively manages plume migration and treats CVOC source areas, and is less expensive to design, implement, operate, and maintain.

### BRIEF SUMMARY OF THE INVENTION

The present invention relates generally to in-situ bioremediation, the use of living organisms to reduce or eliminate environmental hazards resulting from accumulations of toxic chemicals and other hazardous wastes. In-situ bioremediation via enhanced reductive dehalogenation has the potential for cost effectively treating CVOC source areas. Enhanced reductive dehalogenation can also effectively manage CVOC plume migration.

Enhanced reductive dehalogenation ERD involves stimulating the biologically mediated process of reductive dehalogenation, in which chlorine is replaced with hydrogen on the CVOC skeleton, and the CVOC is chemically reduced to a less chlorinated compound. For example, PCE is sequentially dehalogenated to TCE, TCE to DCE, DCE to VC, and ultimately VC to the innocuous end product ethene. Reductive dehalogenation occurs under anaerobic, chemically reducing conditions, typically in the presence of abundant biologically available organic carbon. While co-metabolic reductive dehalogenation may occur (i.e., dehalogenation not beneficial to microflora), the metabolic form (i.e., dehalogenation beneficial to microflora) is associated with more rapid transformation rates, particularly for the more chlorinated CVOC's. During metabolic reductive dehalogenation, certain native microflora use hydrogen as an electron donor and CVOC's as electron acceptors, in the absence of other terminal electron acceptors, in growth-coupled dehalorespiration. During this reaction the microflora use CVOC's as an oxidant substitute to accept the electrons released during metabolism.

Enhanced reductive dehalogenation typically includes injection of a biological stimulant (biostimulant) into the CVOC plume to enhance reductive dehalogenation. The biostimulant is injected to serve as an electron donor for stimulating native microflora to scavenge alternative terminal electron acceptors (oxidants), which can compete with hydrogen and inhibit reductive dehalogenation. The biostimulant should also be biologically degradable to yield fatty acid metabolites, which can be fermented to hydrogen under anaerobic conditions. These fermentation products, most notably hydrogen, drive reductive dehalogenation.

The present invention provides an agent for use as a biostimulant in enhanced reductive dehalogenation with a lower overall cost compared to conventional remedial technologies.

The invention further provides an agent for use as a biostimulant to effectively treat CVOC source areas.

The invention additionally provides a biostimulant to destroy CVOC mass via enhanced reductive dehalogenation instead of transferring it from one environmental media to another.

The invention also provides an agent to make the enhanced reductive dehalogenation less disruptive to facility operations as well as decreasing time to site closure.

### DETAILED DESCRIPTION OF THE INVENTION

The present invention is a Lactose—Brewer's Yeast mixture for use as a biological stimulant for enhancing reductive dehalogenation. The mixture includes Lactose (C₁₂H₂₂O₁₁) and inactive Brewer's Yeast (Saccharomyces). This mixture is a suitable electron donor because it is readily biodegradable such that it can stimulate native microflora to scavenge competing terminal electron acceptors including oxygen, nitrate, oxidized iron, and sulfate by stimulating the respective microbially-mediated biochemical processes of aerobic mineralization, denitrification, ferric iron reduction, and sulfate reduction. In addition, the mixture is ultimately fermentable to hydrogen for driving reductive dehalogenation, and may be manufactured with minimal engineering controls to reduce unit cost. Additionally, the mixture may be delivered to environmental systems in either a high aqueous soluble form for batch injections (large volumes of water containing biostimulant in dissolved form) or in a low aqueous soluble form for borehole injections (smaller volumes of water containing biostimulant in slurry form) or injection socks.

Lactose is a readily biodegradable milk sugar, which can readily stimulate native microflora to scavenge terminal electron acceptors. For example, one gram (g) of Lactose can exert 1.13 g of chemical oxygen demand per liter upon an aqueous environmental system, and Lactose has a five-day biochemical oxygen demand of about 45 grams per liter. Lactose is readily fermented to yield Lactic Acid (CH₃CHOHCO₂H), which can be further fermented to hydrogen, the electron donor driving CVOC reductive dehalogenation. For example, results for eight case studies reported by Regenesis, Inc. (2000) indicate that Lactic Acid, among other materials, stimulated CVOC dehalogenation. Importantly, fermentation reactions are relatively slow, in contrast to direct mineralization reactions in which Lactose serves as an electron donor. Therefore, the Lactose is expected to first scavenge terminal electron acceptors from the environmental media, then be fermented to ultimately yield hydrogen. Lactose also has a moderately high aqueous solubility of about 200 g/L at standard temperature and pressure, so it can be readily distributed throughout groundwater flow systems to treat CVOC impacted zones.

Brewer's Yeast is an additive typically used for enhancing the flavor and texture of food-grade products. The material is a complex micro/macronutrient-enriched growth media, which includes organic and inorganic nutrients and certain vitamins that can support microbial metabolism. In addition to stimulating microflora to scavenge terminal electron acceptors, as does Lactose, the complex organic nutrients are fermentable, and can ultimately yield hydrogen for driving reductive dehalogenation. It has been demonstrated that Yeast Extract amendment stimulated reductive dehalogenation of parent PCE and TCE to 1,2-DCEs in laboratory-scale microcosms within about 180 days. Yeast extract has the same general chemical composition as Brewer's Yeast, however, its cell walls have been lysed. As such, Brewer's Yeast is anticipated to have greater residence time than Yeast Extract in groundwater systems, because of the additional time required for native microflora to break down the cell walls, which consist of long carbohydrate chains composed of polysaccharides.

A 70%_{Weight} Lactose, 30%_{Weight} Brewer's Yeast mixture is the preferred remedial additive blend for most applications. The rationale for the blend ratio is twofold. While Lactose is expected to stimulate reductive dehalogenation, as previously discussed, Lactose is also selected to serve as an electron donor for stimulating microflora to scavenge alternative terminal electron acceptors from groundwater systems and drive conditions anaerobic and chemically reducing. Lactose comprises a bulk of the mixture (70%_{Weight}), given that typical groundwater flow systems are aerobic and chemically reducing. Therefore, a large percentage of Lactose will be spent chemically reducing alternative terminal electron acceptors. Brewer's Yeast is expected to primarily serve as a nutrient/vitamin source as well as a fermentable hydrogen source for driving reductive dehalogenation under the anaerobic, chemically reducing conditions largely stimulated by the Lactose. The blend design also assumes the intact cellular walls of the Brewer's Yeast will decrease its biodegradability rate, because those walls must first be lysed before cellular material is released to become biologically available. During this lag period, the Lactose is expected to stimulate microflora to scavenge alternative terminal electron acceptors from groundwater systems.

The preferred mixture blend design assumes 70%_{weight} Lactose and 30% weight Brewer's Yeast. However, formulations may range from >40%_{weight} Lactose, <60%_{weight} Brewer's Yeast to <85% weight Lactose, >15% weight Brewer's Yeast.

While the preferred mixture consists of 70%_{weight} Lactose, 30%_{weight} Brewer's Yeast, dextrose or sucrose could be substituted for Lactose, given volatility in the Lactose market. In addition, for certain applications such as borehole injections, it might become necessary to coat sugar grains with vegetable oil to reduce their aqueous solubility and prolong their dissolution into groundwater. Based on field and laboratory-scale research, vegetable oil coating delays dissolution of the mixture by as much as a year. In addition, for treatment of CVOC plumes that exist under strictly anaerobic conditions, already entirely depleted of common terminal electron acceptors, it might become necessary to include fatty acids such as Lactic acid in the mixture formulation.

The scope of the invention is not to be considered limited by the above disclosure of the preferred embodiments of the invention. Additional embodiments and advantages will be readily seen by those of ordinary skill in the art in light of the following claims.

1. A biological stimulant for use in bioremediation via enhanced reductive dehalogenation, comprising:
Lactose; and
Brewer's Yeast;
wherein said biological stimulant acts as an electron donor in said reductive dehalogenation.

2. The biological stimulant of claim 1, wherein said Lactose comprises approximately 40% to 85% by weight of said biological stimulant.

3. The biological stimulant of claim 1, wherein said Brewer's Yeast comprises approximately 60% to 15% by weight of said biological stimulant.

4. The biological stimulant of claim 1, wherein said Lactose comprises approximately 70% by weight of said biological stimulant, and wherein said Brewer's Yeast comprises approximately 30% by weight of said biological stimulant.

5. The biological stimulant of claim 1, said biological stimulant further comprising fatty acids.

6. The biological stimulant of claim 1, said biological stimulant further comprising vegetable oil.

7. The biological stimulant of claim 6, said biological stimulant further comprising fatty acids.

8. A biological stimulant for use in bioremediation via enhanced reductive dehalogenation, comprising:
Lactose;
Brewer's Yeast; and
vegetable oil, said vegetable oil coating said Lactose and said Brewer's Yeast;
wherein said biological stimulant acts as an electron donor in said reductive dehalogenation and said vegetable oil coating reduces said biological stimulant's aqueous solubility.

9. The biological stimulant of claim 8, said biological stimulant further comprising fatty acids.

10. The biological stimulant of claim 8, wherein said Lactose comprises approximately 40% to 85% by weight of said biological stimulant.

11. The biological stimulant of claim 8, wherein said Brewer's Yeast comprises approximately 60% to 15% by weight of said biological stimulant.

12. A reductive dehalogenation composition, comprising:
a biodegradable sugar; and
Brewer's Yeast;
wherein said biological stimulant acts as an electron donor in said reductive dehalogenation.

13. The biological stimulant of claim 12, wherein said biodegradable sugar comprises a sugar selected from the group consisting of Lactose, Dextrose, and Sucrose.

14. The biological stimulant of claim 12, wherein said biological stimulant further comprises fatty acids.

15. The biological stimulant of claim 12, wherein said biological stimulant further comprises vegetable oil, and wherein said vegetable oil coats said biodegradable sugar and said Brewer's Yeast.

16. The biological stimulant of claim 15, wherein said biological stimulant further comprises fatty acids.

17. The biological stimulant of claim 12, wherein said Lactose comprises approximately 40% to 85% by weight of said biological stimulant.

18. The biological stimulant of claim 12, wherein said Brewer's Yeast comprises approximately 60% to 15% by weight of said biological stimulant.
